(19) United States
(12) Patent Application Publication
(10) Pub. No.: US 2012/0145189 A1
(43) Pub. Date: Jun. 14, 2012

(54) Portable Self-Heating Steam Generating Device

(76) Inventors: Jeremy F. Knopow, Burlington, WI (US); Jennifer Ruth Harris, Shorewood, WI (US); Keegan J. Trester, Milwaukee, WI (US); Kevin H. Heires, Grand Rapids, MI (US)

(21) Appl. No.: 13/313,834

(22) Filed: Dec. 7, 2011

Related U.S. Application Data
(60) Provisional application No. 61/420,924, filed on Dec. 8, 2010.

(51) Int. Cl. B08B 1/00; A61L 2/18; F22B 1/00

(52) U.S. Cl. 134/6; 122/21; 422/28

(57) ABSTRACT

A portable self-heating device includes a heating cell having a fluid permeable wall defining an interior space of the cell. The device includes an outer cover having a fluid permeable section, and an absorbent layer positioned external to the interior space of the cell. The heating cell and absorbent layer are positioned within a cavity formed by the outer cover such that the absorbent layer contacts the heating cell. A user can activate the portable self-heating device by contacting the device with a fluid (e.g., a gas or a liquid) that reacts exothermically with a fluid activated material located in the interior space of the cell. This raises the temperature of the device. This increase can be sufficient to heat any portion of a liquid adjacent or contacting the device such that the liquid is converted to gas and passes out of the outer cover through the fluid permeable section.

### CROSS-REFERENCES TO RELATED APPLICATIONS

This application claims priority from U.S. Patent Application No. 61/420,924 filed Dec. 8, 2010.

### STATEMENT REGARDING FEDERALLY SPONSORED RESEARCH

Not Applicable.

### BACKGROUND OF THE INVENTION

1. Field of the Invention

This invention relates to a portable self-heating device, and more particularly to a portable self-heating device that can be used to generate steam.

2. Description of the Related Art

Self-heating devices are known. For example, U.S. Pat. No. 6,096,067 describes a disposable thermal body pad comprising one or more thermal packs and a plurality of individual heat cells, which typically comprise an exothermic composition, spaced apart and fixedly attached across the thermal pack.

Steaming devices used to apply steam to objects are known. Steaming devices can apply steam to drapes or garment fabrics to remove wrinkles. Steam-generating warming articles for use on a surface of the human body are also known. See, for example, U.S. Pat. No. 7,652,228. Devices have also been developed for applying steam to a hard surface to assist in the cleaning of the surface. For example, U.S. Patent Application Publication No. 2008/0236635 discloses a steam mop.

While the benefits of steam-generating devices in surface treating are known, these devices often require an electrically-powered boiler to generate steam and an associated pump system to direct the steam towards its intended destination. The boiler and pump often require plugging the device into an AC electrical outlet and therefore, these devices cannot be used where an AC electrical outlet is unavailable. Thus, these devices are not truly portable, and battery operated or rechargeable units are heavy and have limited operational times. Also, all electrical versions require some start up time, usually a few minutes.

What is needed therefore is a portable self-heating device suitable for surface treating applications and a portable self-heating steam generating device that does not require electromechanical steam generating and pumping systems and that can be used to treat soft surfaces, such as fabric, and/or hard surfaces, such as floors, walls, sinks, countertops, bathtubs, toilets, other bathroom fixtures, and inner surfaces of an enclosure.

### SUMMARY OF THE INVENTION

In one aspect, the invention provides a portable self-heating device. The device includes a heating cell, an outer cover, and an absorbent layer. The heating cell has a fluid permeable wall defining an interior space of the cell, and has fluid activated material located in the interior space of the cell. The fluid activated material reacts exothermically when contacted with a fluid that moves through the wall into the interior space of the cell. The fluid can be a gas (e.g., air) or a liquid (e.g., water or a water-based solution), or a two phase system that includes at least one of a gas or a liquid (e.g., suspension of a liquid in a gas, i.e., an aerosol). The outer cover has a fluid permeable section that allows the fluid to pass into a cavity formed by the outer cover. The absorbent layer is positioned external to the interior space of the heating cell. The heating cell and the absorbent layer are positioned within the cavity formed by the outer cover such that the absorbent layer contacts the heating cell. The absorbent layer can assist in transferring fluid that enters the cavity formed by the outer cover to the fluid permeable wall of the heating cell such that the fluid can further travel through the fluid permeable wall of the heating cell and thereby contact the fluid activated material located in the interior space of the cell. The fluid activated material reacts exothermically when contacted with the fluid thereby raising the temperature of the components of the portable self-heating device.

In one form of the device, the fluid selected for use with the portable self-heating device is a gas, such as air. In this form of the device, the outer cover is sealed within a gas impermeable film. A user can activate the portable self-heating device by opening the gas impermeable film such that the gas can pass through the outer cover and through the fluid permeable wall of the heating cell and thereby contact the fluid activated material located in the interior space of the cell. The fluid activated material reacts exothermically when contacted with the gas thereby raising the temperature of the components of the portable self-heating device. The increase in temperature of the components of the portable self-heating device can be sufficient to heat any liquid adjacent to or in contact with one or more of the components of the portable self-heating device such that the liquid is converted to the gas phase. For example, water adjacent to or in contact with one or more of the heated components of the portable self-heating device can be converted to steam that passes out of the device. By “steam”, we mean water vapor, the gaseous phase of water, and also the visible mist of water droplets formed as this water vapor condenses in the presence of cooler air. Water vapor that includes water droplets can be described as wet steam.

In another form of the device, the fluid selected for use with the portable self-heating device comprises a liquid. A user can activate the portable self-heating device by wetting the device with the liquid such that the liquid can pass through the outer cover and through the fluid permeable wall of the heating cell and thereby contact the fluid activated material located in the interior space of the cell. The fluid activated material reacts exothermically when contacted with the liquid thereby raising the temperature of the components of the portable self-heating device. The increase in temperature of the components of the portable self-heating device can be sufficient to heat any portion of liquid adjacent to or in contact with one or more of the components of the portable self-heating device such that the liquid is converted to the gas phase. In one non-limiting example of the device, the fluid selected for use comprises liquid water, and the material reacts exothermically when contacted with the fluid such that at least a portion of the liquid water forms steam that passes through the fluid permeable wall of the heating cell and through the fluid permeable section of the outer cover. Any water adjacent to or in contact with one or more of the heated components of the portable self-heating device can be converted to steam which can pass out of the outer cover by way of the fluid permeable section of the outer cover.

The device is not limited to the production of steam. The fluid activated material can react exothermically when contacted with the fluid such that a temperature rise in the material occurs that is sufficient to convert any component of a liquid mixture adjacent to or in contact with the heated material to a gas that passes through the fluid permeable wall of the heating cell and through the fluid permeable section of the outer cover.

The outer cover can include a first layer bound to a second layer around a periphery of the first layer and a periphery of the second layer. The first layer can comprise all or a part of the fluid permeable section of the outer cover. The second layer can include a fluid impermeable layer. This can be advantageous as the outer cover can be constructed so that any gas (e.g., steam) that passes out of the outer cover will only pass through the first layer due to the second layer including a fluid impermeable layer. In addition, the first layer can include areas having a fluid impermeable layer such that any gas (e.g., steam) that passes out of the outer cover will not pass through these areas of the first layer.

The second layer can be constructed to form a first part of a hook and loop attachment system. This is advantageous as the device may further include a handle constructed to form a second part of the attachment system. The handle can be removably attached to the second layer by way of the first part and the second part of the hook and loop attachment system. The handle can include a base forming the second part of the attachment system, and a grip connected to the base. The grip can be an end section of an elongated shaft. When the device is used in surface treating applications, the heating cell, the outer cover, and the absorbent layer can be configured in a pad form, and the handle facilitates moving the attached pad over the surface being treated. In these surface treating applications, a surface treating material can be incorporated into the pad.

In one form, the portable self-heating device includes a plurality of heating cells. Each of the heating cells can include a fluid permeable wall defining an interior space of each heating cell. Each heating cell can include fluid activated material located in the interior space of each heating cell. The material reacts exothermically when contacted with a fluid that moves through the wall of each heating cell into the interior space of each heating cell. The absorbent layer can be configured as a web that attaches each of the heating cells together in a spaced apart relationship. The absorbent layer can be formed by heat sealing two absorbent layers around the fluid activated material to create the heating cells. In one non-limiting example form, the two layers are heat sealed to create a heat seal structure resembling a window frame with the heating cells equally spaced between the heat sealed areas of the absorbent layer. The heat sealed areas of the absorbent layer can promote the flow/dispersal of a liquid (e.g., water) from a central dosing point of the liquid along the heat sealed areas of the absorbent layer via capillary action to the heating cells speeding up the reaction time with the fluid activated material in the heating cells.

In another aspect, the invention provides a portable self-heating device. The device includes a heating cell having a fluid permeable wall defining an interior space of the cell and having fluid activated material located in the interior space of the cell. The material reacts exothermically when contacted with fluid that moves through the wall into the interior space of the cell. The device further includes an outer cover having a fluid permeable section, and an absorbent layer. The absorbent layer and the heating cell are positioned within a cavity formed by the outer cover such that the absorbent layer contacts the heating cell. The device further includes a handle removably attached to the outer cover. The handle includes a source of fluid and a fluid conduit in fluid communication with the source of fluid and a nozzle on a surface of the handle adjacent the outer cover. The handle includes a fluid delivery system for moving fluid from the source of fluid, through the fluid conduit, through the nozzle, through the cover, and into the absorbent layer. The heating cell, the outer cover, and the absorbent layer can be configured in a pad form. When the fluid delivery system moves the fluid from the source of fluid and into the absorbent layer, the fluid activated material reacts exothermically when contacted with the fluid thereby raising the temperature of the components of the pad. The chemistry responsible for the exothermic reaction can be provided in the fluid activated material and/or in the fluid provided by the fluid delivery system of the handle. It can be beneficial to provide the chemistry responsible for the exothermic reaction in the fluid provided by the fluid delivery system of the handle such that the pad can be formed as a durable reusable pad that stays on the handle for repeated uses. Alternatively, the fluid activated chemistry can be dosed independent of the fluid, with the fluid being added in a different step such as running the pad under water. In this version of the invention, the exothermic material is dosed as opposed to the fluid to cause the exothermic reaction.

The fluid can be a gas (e.g., air) or a liquid (e.g., water or a water-based solution), or a two phase system that includes at least one of a gas or a liquid (e.g., suspension of a liquid in a gas, i.e., an aerosol). In one version of the device, the fluid selected for use with the portable self-heating device comprises a liquid. When the fluid delivery system moves the fluid from the source of fluid and into the absorbent layer, the fluid activated material reacts exothermically when contacted with the fluid thereby raising the temperature of the components of the pad. The increase in temperature of the pad can be sufficient to heat any portion of liquid adjacent to or in contact with one or more of the components of the pad such that the liquid is converted to the gas phase. In one non-limiting example of the device, the fluid selected for use comprises liquid water, and the material reacts exothermically when contacted with the fluid such that at least a portion of the liquid water forms steam that passes through the fluid permeable wall of the heating cell and through the fluid permeable section of the outer cover of the pad. Any water adjacent to or in contact with one or more of the heated components of the pad can be converted to steam which can pass out of the outer cover by way of the fluid permeable section of the outer cover. The handle can include a base attached to the outer cover, and the base can include at least one throughhole for passage of steam through the base.

The fluid delivery system can include a variable volume pump chamber in fluid communication with the source of fluid and the fluid conduit. The variable volume pump chamber moves fluid from the source of fluid, through the fluid conduit, and through the nozzle. The volume of the pump chamber can be varied by an actuator on the handle. In one non-limiting example form, the actuator is connected to a bellows pump chamber.

The fluid delivery system can include a valve having a closed position in which fluid cannot move from the source of fluid, through the fluid conduit, and through the nozzle, and having an open position in which fluid can move from the source of fluid, through the fluid conduit, and through the nozzle. The valve can control gravity feed of fluid from the source of fluid, or the valve may be the valve on an aerosol can when an aerosol can is the source of fluid.

The outer cover of the pad can include a first layer bound to a second layer around a periphery of the first layer and a periphery of the second layer. The first layer can comprise all or a part of the fluid permeable section of the outer cover. The second layer can include a fluid impermeable layer. This can be advantageous as the outer cover can be constructed so that any gas (e.g., steam) that passes out of the outer cover will only pass through the first layer due to the second layer including a fluid impermeable layer. In addition, the first layer can include areas having a fluid impermeable layer such that any gas (e.g., steam) that passes out of the outer cover will not pass through these areas of the first layer.

The second layer can be constructed to form a first part of a hook and loop attachment system. This is advantageous as the handle can constructed to form a second part of the attachment system. The handle can be removably attached to the second layer by way of the first part and the second part of the hook and loop attachment system. The handle can include a base forming the second part of the attachment system, and a grip connected to the base. The grip can be an end section of an elongated shaft. When the device is used in surface treating applications, the handle facilitates moving the attached pad over the surface being treated. In these surface treating applications, a surface treating material can be incorporated into the pad.

In one form, the pad of the portable self-heating device includes a plurality of heating cells. Each of the heating cells can include a fluid permeable wall defining an interior space of each heating cell. Each heating cell can include fluid activated material located in the interior space of each heating cell. The material reacts exothermically when contacted with a fluid that moves through the wall of each heating cell into the interior space of each heating cell. The absorbent layer can be configured as a web that attaches each of the heating cells together in spaced apart relationship. The absorbent layer can be formed by heat sealing two absorbent layers around the fluid activated material to create the heating cells. In one non-limiting example form, the two layers are heat sealed to create a heat seal structure resembling a window frame with the heating cells equally spaced between the heat sealed areas of the absorbent layer. The heat sealed areas of the absorbent layer can promote the flow/dispersal of a liquid (e.g., water) from a central dosing point of the liquid along the heat sealed areas of the absorbent layer via capillary action to the heating cells speeding up the reaction time with the fluid activated material in the heating cells. In one form, the second layer of the outer cover includes an aperture, and the nozzle directs fluid through the aperture and into the central dosing point of the absorbent layer.

In yet another aspect, the invention provides a method for cleaning and/or sanitizing and/or disinfecting a surface. In the method, a pad including at least one heating cell, the outer cover, and the absorbent layer is contacted with a fluid to form a wetted device, and the wetted device is placed on or adjacent the surface to contact the surface with a gas produced by the wetted device. In one non-limiting form, the fluid comprises liquid water, and the material reacts exothermically when contacted with the fluid such that at least a portion of the liquid water forms steam that passes through the fluid permeable wall of the heating cell and through the fluid permeable section of the outer cover. Non-limiting examples of the surfaces that can be treated include floors, walls, countertops, sinks, bathtubs, toilets, bathroom fixtures, and inner surfaces of an enclosure.

In still another aspect, the invention provides a portable self-heating steam generating device. The portable self-heating steam generating device includes a heating pouch having a water permeable wall defining an interior space of the pouch. The heating pouch has a water activated material located in the interior space of the pouch wherein the material reacts exothermically when contacted with water that moves through the wall into the interior space of the pouch. The portable self-heating steam generating device further includes an outer cover having a gas permeable section and a water permeable section, and an absorbent layer. The heating pouch and the absorbent layer are positioned within a cavity formed between the bound first and second layers such that the second layer contacts the heating pouch. In one form of the device, the outer cover comprises a first layer bound to a second layer around a periphery of the first layer and a periphery of the second layer. In another form of the device, the second layer is constructed to form a first part of a hook and loop attachment system. In yet another form of the device, a surface treating material is incorporated into the device. In still another form of the device, the surface treating material is attached to a surface of the absorbent layer or a surface of the second layer. In yet another form of the device, the surface treating material is positioned between the absorbent layer and the second layer. In still another form of the device, the surface treating material comprises a surfactant or a fragrance, or an odor eliminator, or a wrinkle releaser. The fragrance, or an odor eliminator, or a wrinkle releaser may be encapsulated.

In yet another form of the device, a handle is constructed to form a second part of the hook and loop attachment system, and the handle is attached to the second layer. In still another form of the device, the handle comprises a base forming the second part of the hook and loop attachment system, and the handle includes a grip connected to the base. In yet another form of the device, the grip is an end section of an elongated shaft of the handle.

In yet another aspect, the invention provides a portable self-heating steam generating device. The portable self-heating steam generating device includes a heating pouch having a water permeable wall defining an interior space of the pouch and having water activated material located in the interior space of the pouch wherein the material reacts exothermically when contacted with water that moves through the wall into the interior space of the pouch. The portable self-heating steam generating device further includes an outer cover having a gas permeable section and a water permeable section, and an absorbent layer wherein the absorbent layer and the heating pouch are positioned within a cavity formed by the outer cover such that the absorbent layer contacts the heating pouch. The portable self-heating steam generating device further includes a handle attached to the pad. The handle includes a source of water and a fluid conduit in fluid communication with the source of water and a nozzle on a surface of the handle adjacent the pad. The handle further includes a water delivery system for moving water from the source of water, through the fluid conduit, through the nozzle, through the cover, and into the absorbent layer.

In one form of the device, the handle includes at least one additional nozzle in fluid communication with the source of water. In yet another form of the device, the water delivery system includes a variable volume pump chamber in fluid communication with the source of water and the fluid conduit wherein the variable volume pump chamber moves water from the source of water, through the fluid conduit, and through the nozzle. In still another form of the device, the volume of the pump chamber is varied by an actuator on the handle. In yet another form of the device, the water delivery system includes a valve having a closed position in which water cannot move from the source of water, through the fluid conduit, and through the nozzle. The valve also has an open position in which water can move from the source of water, through the fluid conduit, and through the nozzle. In still another form of the device, the water delivery system further includes an actuator on the handle for moving the valve into the closed position or the open position. In yet another form of the device, the outer cover comprises a first layer bound to a second layer around a periphery of the first layer and a periphery of the second layer. In still another form of the device, the second layer is constructed to form a first part of a hook and loop attachment system. In yet another form of the device, the handle is constructed to form a second part of the hook and loop attachment system, and the handle is removably attached to the second layer.

In yet another aspect, the invention provides a method for cleaning and/or sanitizing and/or disinfecting a surface. In the method, the portable self-heating steam generating device is wetted to form a wetted device, and the wetted device is placed on or adjacent the surface to contact the surface with steam produced by the wetted device. The surface can be selected from floors, walls, countertops, sinks, bathtubs, toilets, bathroom fixtures, and inner surfaces of an enclosure.

In still another aspect, the invention provides a method for treating a soft surface such as fabric. In the method, the portable self-heating steam generating device is wetted to form a wetted device, and the wetted device is placed on or adjacent the soft surface to contact the soft surface with steam produced by the wetted device. The soft surface can be fabric that is part of a garment. The soft surface can be fabric that is part of a furniture piece. The soft surface can be fabric that is part of a drapery.

One advantageous feature of the portable self-heating steam generating device is the absorbent layer. Current water activated heaters use a bag or container to which the end user adds water to activate the heat. The heat is then transferred to adjacent objects through conduction. In the present invention, fluid, such as water, is captured in the absorbent layer directly in contact with the heating cells, allowing for prolonged use without carrying around liquid water. In one embodiment, the delivery of fluid (e.g., water) to the heating cells is controlled by the end user. This gives the user the ability to control the amount of heat produced and the longevity of the fluid activated material in the heating cells. In some sense, it is an “on/off” switch for the heat. This is highly desirable by end users and is a feature not in other heater devices, where all of the necessary water necessary to activate the entire heater is added at once.

Some suitable non-limiting uses for a portable self-heating steam generating device according to the invention include: (i) handheld steam disinfecting/cleaning of hard surfaces; (ii) floor steam disinfecting/cleaning; (iii) fabric, furniture, drapery steaming; (iii) clothing steaming; (iv) carpet stain removal; (v) a heater element supplied as a cartridge to go into a hard molded device, e.g., a cylindrical device where a user inserts a heater cartridge into the center and rolls it across a surface; and (vi) a heat/steam energy source such as a mini steam engine.

These and other features, aspects, and advantages of the present invention will become better understood upon consideration of the following detailed description, drawings and claims.

### BRIEF DESCRIPTION OF THE DRAWINGS

FIG. 1 is a perspective view of a three layer self-heating steam generating pad used in one embodiment of a self-heating steam generating device of the present invention.

FIG. 2 is a cross-sectional view of the three layer self-heating steam generating pad of FIG. 1 taken along line 2-2 of FIG. 1.

FIG. 2A is a cross-sectional view, similar to FIG. 2, of another embodiment of a self-heating steam generating pad according to the invention.

FIG. 2B is a cross-sectional view, similar to FIG. 2, of yet another embodiment of a self-heating steam generating pad according to the invention.

FIG. 3 is a perspective view showing how a pad of FIG. 1 can be mounted on a handle.

FIG. 4 is a perspective view of the pad of FIG. 1 mounted on such a handle.

FIG. 5 is a perspective view of a mop-type handle suitable for mounting a pad of FIG. 1.

FIG. 6 is a perspective view of a pad of FIG. 1 mounted on yet another handle that provides for water delivery to the pad.

FIG. 7 is a perspective view showing how a pad of FIG. 1 can be mounted on the handle of FIG. 6.

FIG. 8 is a schematic representation depicting one example water delivery system that can be used in the handle of FIGS. 6 and 7 and 12.

FIG. 9 is a perspective view of a pad of FIG. 1 mounted on another mop-type handle that provides for water delivery to the pad.

FIG. 10 is a top view of the pad of FIG. 2B with the top attachment layer removed.

FIG. 11 is a top view of the pad of FIG. 2B, similar to FIG. 10, with the top attachment layer shown.

FIG. 12 is a perspective view of the pad of FIG. 2B mounted on another embodiment of a handle.

FIG. 13 is a side view of the fluid delivery system of the handle of FIG. 12.

FIG. 14 shows a perspective view of the handle of FIG. 12 in one position used for cooling an attached pad (the pad not being shown in FIG. 14).

FIG. 15 is a perspective view of a pad of FIG. 2B mounted on yet another mop-type handle that provides for fluid delivery to the pad.

FIG. 16 shows a perspective view of the mop-type handle of FIG. 15 in one storage position.

FIG. 17 is a perspective view of a pad of FIG. 2B mounted on a brush-type handle.

Like reference numerals will be used to refer to like parts from Figure to Figure in the following description of the drawings.

### DETAILED DESCRIPTION OF THE INVENTION

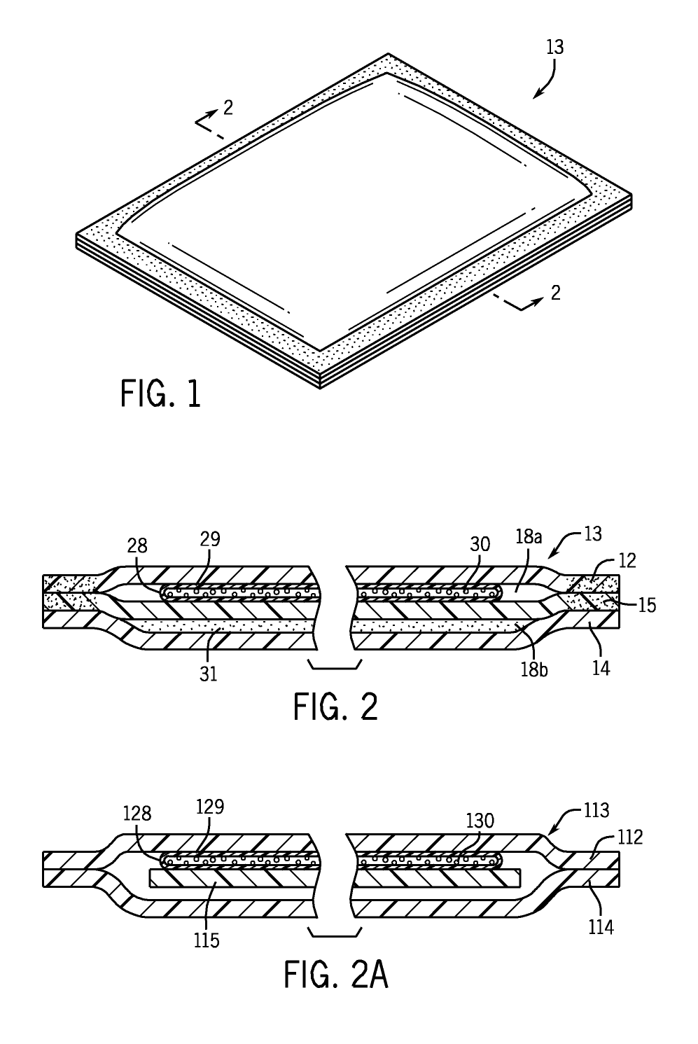

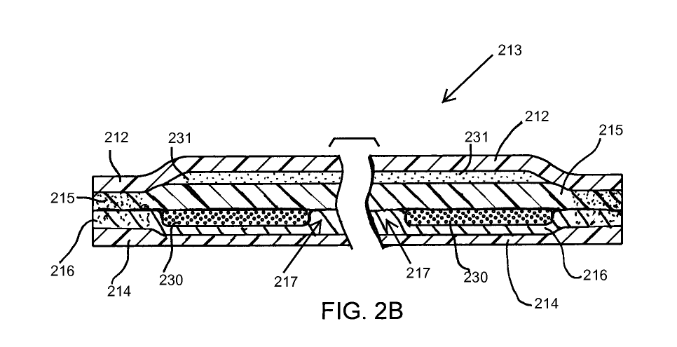

FIGS. 1 and 2 show an example embodiment of a three layer self-heating steam generating pad 13 used in one embodiment of a self-heating steam generating device of the present invention. The three-layer pad 13 includes outer layers 12 and 14. The layer 12 is gas-permeable and water-permeable, and optionally abrasive if the pad 13 is used for cleaning surfaces. The middle layer 15 is sandwiched between the outer layers 12 and 14. The middle layer 15 is water absorbent. The layers 12, 14 and 15 are sealed around the periphery of the layers 12 and 14 and 15 to define a cavity 18a between layers 12 and 15 and a cavity 18b between layers 14 and 15. The layers 12 and 14 form an outer cover for the pad 13.

In non-limiting example forms, the layers 12, 14 and 15 can be a sheet of woven or non-woven fabric, textile-like material, foamed sheet, or plastic sheeting, or combinations thereof. One or more of layers 12, 14 and 15 may be of a porous nature, so as to allow the passage of gas and/or water and/or an aqueous cleaning solution. The pad 13 may also be constructed of at least one water-impervious layer, such as a continuous polyethylene sheet. The outer edges of the layers 12, 14 and 15 are preferably bonded or joined together by stitching, heat welding, sonic welding, adhesive or other means. Preferably, the outer edges of the layers 12, 14 and 15 are bonded together around at least half their periphery, and most preferably, the outer edges of the layers 12, 14 and 15 are bonded together around their entire periphery, the joined adjacent layers forming the sealed cavities 18a, 18b.

When the pad 13 is intended for cleaning hard surfaces, the layer 12 which faces the surface to be cleaned may include polymeric fibers in a shape suitable for providing abrasion. The polymeric fibers in the layer 12 are generally arranged to form an open, porous gas-permeable and water-permeable structure. All of the layer 12 may be gas-permeable and water-permeable, or certain sections of the layer 12 may be gas-permeable and water-permeable. Also, the same section of the layer 12 may comprise both the gas-permeable section and the water-permeable section. The layer 12 is capable of providing a scrubbing function, rather than just polishing, wiping or drying functions. In one form, the layer 12 has a basis weight of about 10 g/m² to about 300 g/m². In a non-limiting example embodiment, the layer 12 can be made of polyester/acrylic resin material such as 100% polyester fibers bonded together with an acrylic resin binder. One suitable abrasive layer is the material sold as Matador Grade RD3370-2 (Matador Converters Co. Ltd., Canada), which is 100% polyester fibers bonded together with an acrylic resin binder. The abrasiveness of the abrasive layer can be varied depending on the intended use of the product. For example, the abrasiveness can be increased by providing elevated and depressed regions in the surface of the layer 12. Also, the fiber materials, fiber length, fiber cross-section, fiber diameter, layer basis weight, etc. may all vary depending on the desired abrasiveness of the abrasive layer.

The layer 14 can be constructed to be suitable for forming a hook and loop type attachment system with a corresponding surface on a mounting handle. In a non-limiting example embodiment, the layer 14 could be made of at least partially synthetic non-woven material mounted on a synthetic extrusion film. The outer surface of the layer 14 can be the non-woven material which functions as the loop material for the hook and loop type attachment system (such as a Velcro™ assembly system) without the need for a separate loop strip. In one form, the layer 14 is a polyester spunlaced nonwoven material mounted on a polyethylene extrusion film (about 25 micrometers thick), such as sold by Ahlstrom Grade 26032 (Ahlstrom Windsor Locks LLC, CT, USA). The layer 14 may be water permeable or water impermeable depending on the expected use of the pad. For example, the extrusion film of the layer 14 can prevent the passage of water and gas such that steam generated by the pad 13 only exits the pad 13 at the layer 12. This serves to direct the steam only at the surface being treated and not toward the user. The extrusion film of the layer 14 can also perform a heat barrier function, that is, the film can limit heat transfer toward the outer surface of the layer 14.

The layer 15 (which is in the middle in a three-layer structure) can be made of at least a partially synthetic non-woven material. One suitable porous middle layer is the material sold as Matador Grade FF0305, which is a 100% polyester nonwoven material. Another suitable porous absorbent middle layer is the material sold as Matador Grade RD3370-2, which is 100% polyester fibers bonded together with an acrylic resin binder. Another suitable material for layer 15 is an absorbent at least partially synthetic material sold as Ahlstrom Grade 12236, which is a non-woven fabric formed from a pulp/synthetic mix.

In the cavity 18a, there is positioned a heating pouch 28 having a gas permeable and water permeable wall 29 defining an interior space of the pouch 28. A water activated material 30 is located in the interior space of the pouch 28. The material 30 reacts exothermically when contacted with water that moves through the layer 12 and/or the layer 15 and through the wall 29 into the interior space of the pouch 28. The wall 29 can be constructed of a porous film capable of forming a gas permeable and water permeable pouch using mechanical means and/or heat. Non-limiting examples of such films are polyethylene, polypropylene, nylon, polyester, polyvinyl chloride, polyvinylidene chloride, polyurethane and rubber.

The water activated material 30 can include an electrolyte-producing salt, such as sodium chloride, and a supercorroding alloy such as an alloy including magnesium and 5 atomic percent iron. Water wets the water activated material which generates heat through an exothermic reaction. The water that activates the water activated material 30 can be provided in liquid that is essentially water, or the water can be provided in a liquid that is an aqueous solution, such as a saline solution which may react more favorably with the water activated material 30.

Alternatively, the water activated material 30 can include (i) a basic component, such as calcium hydroxide, potassium hydroxide, sodium acetate, sodium benzoate, potassium ascorbate, calcium oxide, lithium oxide, sodium oxide, potassium oxide, rubidium oxide, cesium oxide, magnesium oxide, strontium oxide, and barium oxide; and (ii) an acidic component, such as aluminum chloride, zinc chloride, titanium tetrachloride, ferrous chloride, ferric nitrate, and phosphorus pentoxide. Water wets the water activated material which generates heat through an exothermic reaction.

Alternatively, the water activated material 30 can include aluminum powder and calcium oxide powder. Water wets the water activated material which generates heat through an exothermic reaction.

Alternatively, the water activated material 30 can include magnesium and iron and an oxidizing agent, such as calcium nitrate, calcium hydroxide, sodium chloride, sodium nitrate, sodium nitrite, iodates, and potassium permanganate. Water wets the water activated material which generates heat through an exothermic reaction.

Alternatively, the water activated material 30 can include iron powder and a carbonaceous material, such as activated carbon, and an electrolyte-producing salt, such as sodium chloride. Water wets the water activated material which generates heat through an exothermic reaction.

The heat generated by the exothermic reaction of the water and the water activated material 30 can be used to heat water to produce steam that flows out of the pad 13 and onto a surface being treated. The water that is converted to steam can be: (i) present in the pouch 28 due to movement of water into the pouch 28; and/or (ii) absorbed in the layer 12; and/or (iii) absorbed in the layer 15; and/or (iv) absorbed in the layer 14; and/or (v) present in the cavity 18a; and/or (vi) present in the cavity 18b; and/or (vii) present in or on the surface being treated by the pad 13. The steam that flows out of the pad 13 may activate a heat activated indicator that changes color upon heating. The heat activated indicator may be located on either or both of the layers 12 and 14. Suitable heat activated materials to act as an activation cue include liquid crystals or leuco dyes.

When the pad 13 is used for cleaning surfaces, it can be beneficial to incorporate a surface treating material 31 into the pad 13. Non-limiting examples of a surface treating material include one or more of the following: anionic surfactants, nonionic surfactants, cationic surfactants, amphoteric/zwitterionic surfactants, detergent builders, chelating agents, rinse aids, surface modifying anti-resoiling agents, inorganic or organic pH buffering agents, solid hydrotroping agents, dyes, fragrances, odor eliminators and wrinkle releasers. Where the surface treating material includes more than one chemical component, it is desirable that the components be combined and processed to form a relatively homogeneous mixture prior to incorporation into the pad 13. The surface treating mixture can be pre-formed into solid particles or a solid surface treating block that inserted into the cavity 18b the pad. The surface treating mixture may also be adhered to the layer 12 and/or layer 14 and/or the layer 15. The surface treating mixture can dissolve when contacted with water to produce a cleaning solution that flows out of the pad 13 and onto a surface being treated. The heat generated by the pad 13 may also aid dissolution and/or activation of the surface treating material 31. The layer 12 can be used to scrub the surface that receives the surface treating (e.g., cleaning) solution from the pad 13. As used herein, surface treating materials are not limited to materials that contact a surface. For example, a fragrance may treat atmospheric areas adjacent or near a surface.

In an alternative version of the pad 13, the heating pouch 28 has a gas impermeable wall 29 defining an interior space of the pouch 28. An air activated material is located in the interior space of the pouch 28. The pouch is broken open to allow air to enter the interior space of the pouch 28 such that the air activated material reacts exothermically when contacted with air that moves into the interior space of the pouch 28. An example of an air activated material is a material comprising iron powder, a small amount of water, vermiculite, active carbon, and sodium chloride. The material gives off heat when air oxidizes the iron.

In an alternative version of the pad 13, the heating pouch 28 has a gas permeable wall 29 defining an interior space of the pouch 28. An air activated material is located in the interior space of the pouch 28. A gas impermeable packaging film is provided around the pad 13, and the film is opened such that the air activated material reacts exothermically when contacted with air that moves into the interior space of the pouch 28.

Turning to FIG. 2A, there is shown another embodiment of a self-heating steam generating pad 113 used in another embodiment of a self-heating steam generating device of the present invention. The pad 113 includes outer layers 112 and 114. The layer 112 is gas-permeable and water-permeable, and optionally abrasive if the pad 113 is used for cleaning surfaces. A middle layer 115 is water absorbent. The layers 112 and 114 are sealed around the periphery of the layers 112 and 114 to form an outer cover that defines a cavity between layers 112 and 114. The layers 112, 114, 115 can comprise the same materials as layers 12, 14, 15 respectively as described above.

In the cavity of the outer cover of the pad 113, there is positioned a heating pouch 128 having a gas permeable and water permeable wall 129 defining an interior space of the pouch 128. A water activated material 130 is located in the interior space of the pouch 128. The material 130 reacts exothermically when contacted with water that moves through the layer 112 and/or the layer 115 and through the wall 129 into the interior space of the pouch 128. The pouch 128 and the water activated material 130 can comprise the same materials as the pouch 28 and water activated material 30 described above. Alternatively, an air activated material is located in the interior space of the pouch 28 as in pad 13.

Comparing pad 13 to pad 113, it can be appreciated that in pad 13 the absorbent layer 15 is bound to the layers 12 and 14, whereas in pad 113, the absorbent layer 115 is not bound to the layer 112 and 114. In other words, absorbent layer 115 can move around between the layer 114 and the heating pouch 128, or between the layer 112 and the heating pouch 128. Under certain conditions, it may be easier to seal up the pad 113 without the absorbent layer 115 involved in the sealing stack. Also, one or more absorbent layers 115 can be located between the layer 114 and the heating pouch 128, or between the layer 112 and the heating pouch 128. Additional absorbent layers 115 between the heating pouch 128 and layer 112 can help absorb moisture, etc. off the surface being treated. In one example form, two absorbent layers 115 are positioned on either side of the heating pouch 128.

Turning to FIG. 2B, there is shown yet another embodiment of a self-heating steam generating pad 213 used in another embodiment of a self-heating steam generating device of the present invention. The pad 213 includes outer layers 212 and 214. The layer 212 is gas-permeable and water-permeable, and optionally abrasive if the pad 213 is used for cleaning surfaces. First middle layer 215 and second middle layer 216 are water absorbent. The layers 212 and 214 are sealed around the periphery of the layers 212 and 214 to form an outer cover that defines a cavity between layers 212 and 214.

Looking at FIGS. 2A, 10 and 11, a water activated material 230 is located in four heating cells 219 created by heat sealing the first middle layer 215 and the second middle layer 216 together along areas 217 of the first middle layer 215 and the second middle layer 216. The material 230 reacts exothermically when contacted with water that moves through the layer 212 and/or the middle layers 215, 216 into the interior space of each heating cell 219. The water may comprise a part of a fluid such as saline.

Still referring to FIGS. 2A, 10 and 11, the layer 214 can be constructed to be suitable for forming a hook and loop type attachment system with a corresponding surface on a mounting handle. In a non-limiting example embodiment, the layer 214 could be made of at least partially synthetic non-woven material mounted on a synthetic extrusion film. The outer surface of the layer 214 can be the non-woven material which functions as the loop material for the hook and loop type attachment system (such as a Velcro™ assembly system) without the need for a separate loop strip. The extrusion film of the inner surface of the layer 214 can prevent the passage of water and gas such that steam generated by the pad 213 only exits the pad 213 at the layer 212. Looking at FIG. 11, the layer 214 can include a central aperture 221 that allows dosing of a fluid through the aperture 221 and onto a central fluid distribution site 225 that is a part of the heat sealed areas 217 of the first middle layer 215 and the second middle layer 216. Fluid that contacts the fluid distribution site 225 travels out in the heat sealed areas 217 toward the periphery of the pad 213.

One non-limiting example of the pad 213 was constructed as follows. For the layer 212, a polyester abrasive available from Matador Converters Co. Ltd. was used. This material is fluid permeable. For the layer 214, a laminated substrate of N35 loop polyester with a twenty micron polypropylene film backing adhesively bonded with polyurethane hot-melt moisture-cured adhesive was used. This material is available from Aplix Inc., Charlotte, N.C., USA. For the first middle layer 215 and the second middle layer 216, a blend of cellulose and synthetic binder fibers available from Ahlstrom was used. This material is absorbent. For the water activated material 230, a material was used having the following ratio of ingredients: a 9 gram of sample of powder comprised a mixture of 7.5 grams magnesium/5 atomic weight percent iron supercorroding alloy, 0.7 grams inert filler, 0.5 grams NaCI, and 0.3 grams antifoaming agents. Biodegradable materials can be selected for the layers 212, 213, 214, 215 and 216, and the water activated material 230.

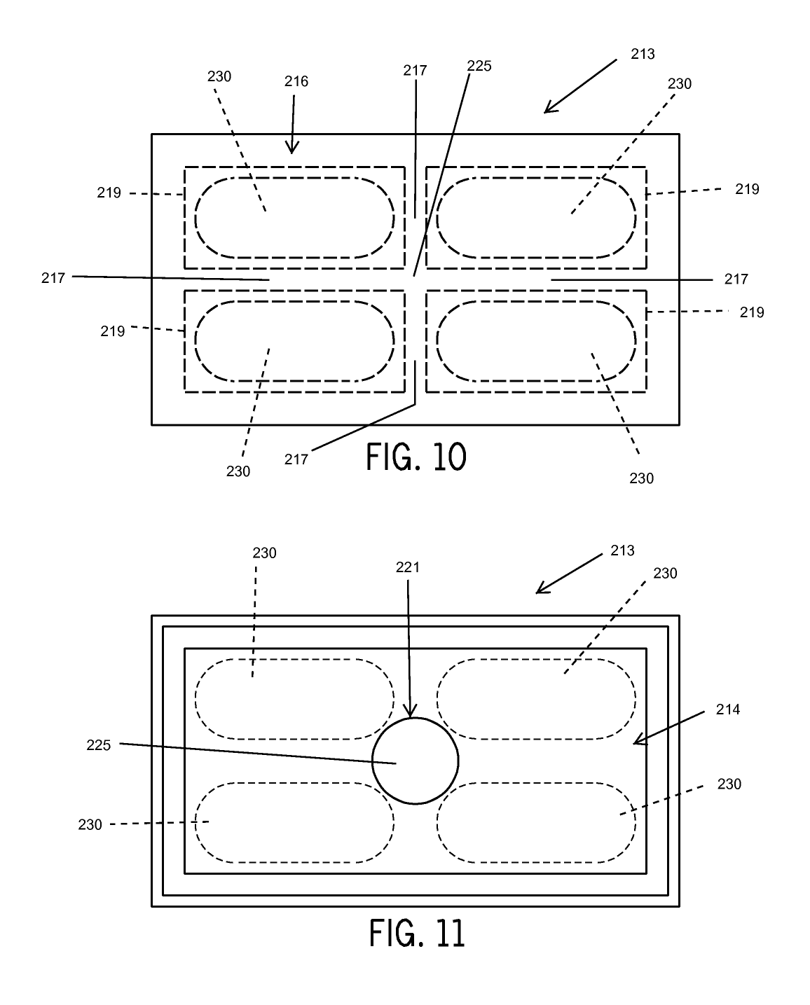

The absorbent first middle layer 215 and the absorbent second middle layer 216 were heat sealed around four one gram samples of the water activated material 230 to create the internal heater element having four heating cells 219 (see FIG. 10). A “window pane” seal structure was created by the heat seal areas 217 of the first middle layer 215 and the second middle layer 216 and by the sealed periphery of the first middle layer 215 and the second middle layer 216. This heat seal structure was developed to promote the flow/dispersal of the fluid (e.g., water) used to activate the water activated material 230. From the central fluid distribution site 225, the fluid (e.g., water) runs along the heat sealed areas 217 via capillary action to the water activated material 230 speeding up the reaction time.

The example of the pad 213 that was constructed in this manner included four grams total of the water activated material 230 (i.e., one gram per cell). Twelve microliters of fragrance was applied to the absorbent layer 215 over each heating cell 219 (total 48 μl). The layer 212 (the cleaning layer in pad 213) was heat sealed around its periphery to the middle layer 215, and the layer 214 (the attachment layer in pad 213) was heat sealed around its periphery to the second middle layer 216.

Fluid (water in this example) is dosed onto the central fluid distribution site 225 of the first middle layer 215 and the second middle layer 216 through aperture 221 in the layer 214. The polypropylene film backing of the inner surface of the layer 214 keeps water from wicking away from heating cells 219 and also helps direct steam created by heating of the water down and out of pad 213 through the layer 212 onto the surface being cleaned.

Another non-limiting example of a pad similar to the pad 213 was constructed with eight heating cells. When viewed from the top or bottom, this pad appears as two of the pads 213 arranged in an abutting side by side relationship. In this embodiment, the same materials were selected for the layer 212, the layer 214, the first middle layer 215, the second middle layer 216, and the water activated material 230. The absorbent first middle layer 215 and the absorbent second middle layer 216 were heat sealed around eight samples of the water activated material 230 to create the internal heater element having eight heating cells 219 (similar to the four heating cells 219 in FIG. 10). A “window pane” seal structure was created by the heat seal areas 217 of the first middle layer 215 and the second middle layer 216 and by the sealed periphery of the first middle layer 215 and the second middle layer 216. This heat seal structure was developed to promote the flow/dispersal of the fluid (e.g., water) used to activate the water activated material 230. From a central fluid distribution site, the fluid (e.g., water) runs along the heat sealed areas 217 via capillary action to the water activated material 230 speeding up the reaction time.

The example of the pad 213 that was constructed in this manner included eight grams total of the water activated material 230 (i.e., one gram per cell). Twelve microliters of fragrance was applied to the absorbent layer 215 over each heating cell 219 (total 96 μl). The layer 212 (the cleaning layer in pad 213) was heat sealed around its periphery to the middle layer 215, and the layer 214 (the attachment layer in pad 213) was heat sealed around its periphery to the second middle layer 216.

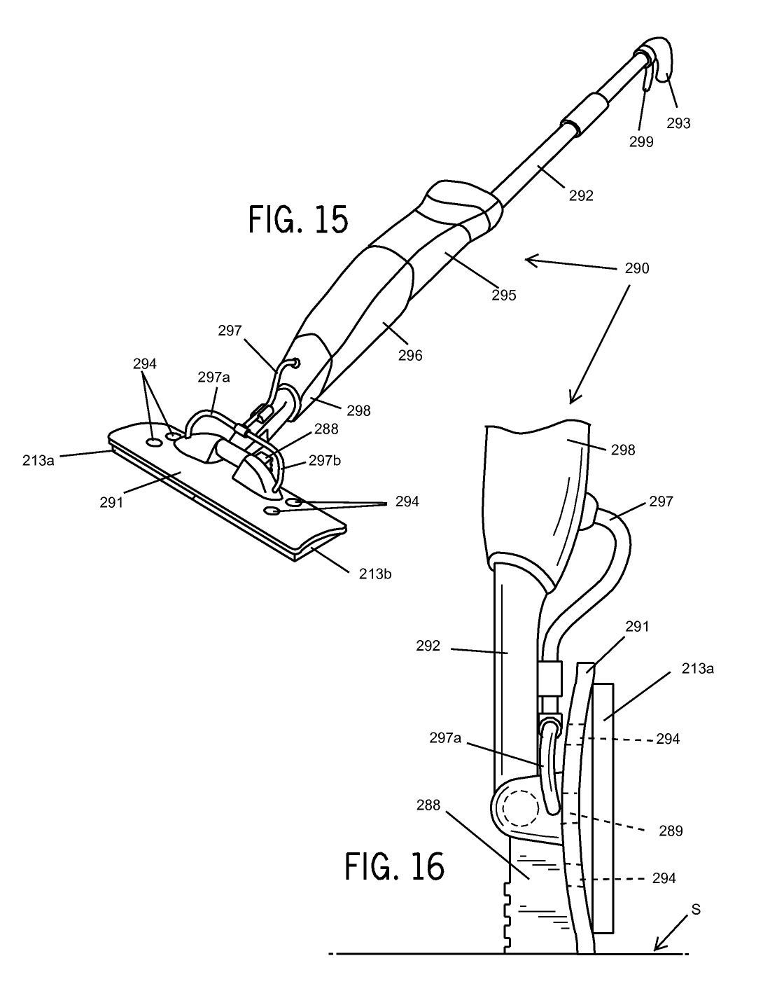

Fluid (water in this example) is dosed onto the central fluid distribution site of the first middle layer 215 and the second middle layer 216 through a central aperture (similar to aperture 221) in the layer 214. The polypropylene film backing of the inner surface of the layer 214 keeps water from wicking away from heating cells 219 and also helps direct steam created by heating of the water down and out of pad 213 through the layer 212 onto the surface being cleaned. In this embodiment, four additional holes around the layer 214 allow steam/water vapor to escape through the layer 214 and hence through corresponding holes 294 in a mop head (see description of FIGS. 15 and 16 below) cueing users that the pad 213 is activated. Steam/heat activation lasted for approximately ten minutes.

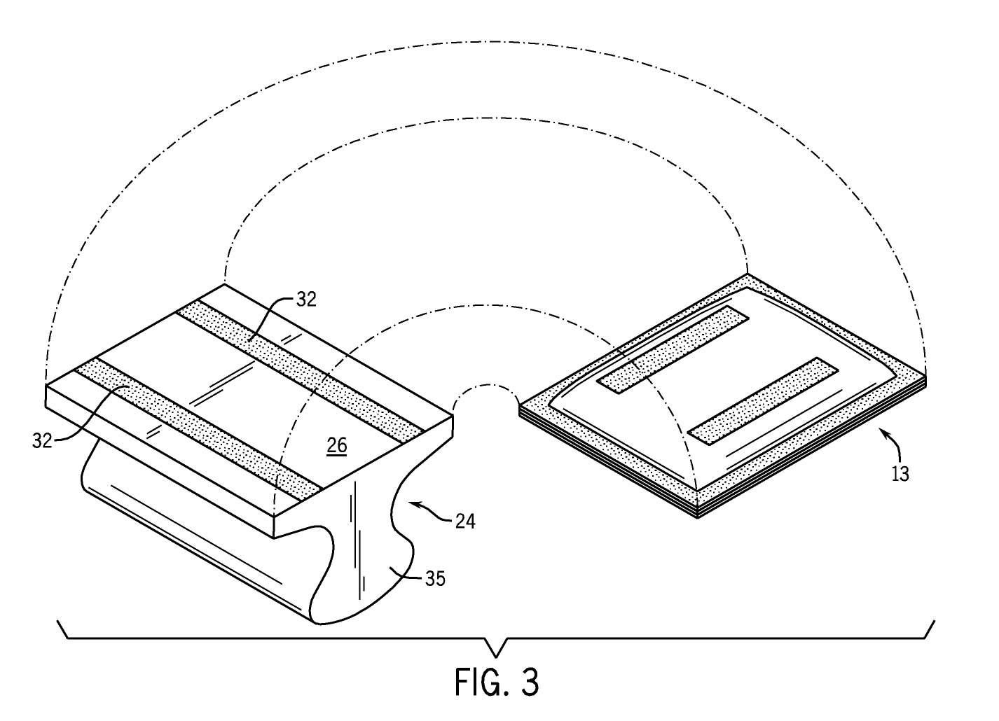

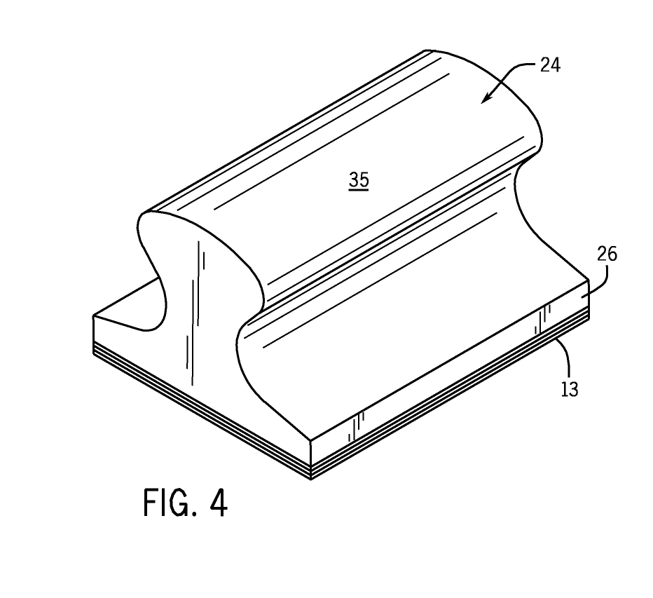

Referring next to FIGS. 3 and 4, the pad 13 (or pads 113, 213) of the present invention can be used in combination with a mounting handle 24. The outer layer 14 of the pad 13 can be constructed to form a first part of a hook and loop attachment system, and strips 32 on the base 26 of the handle 24 can form a second part of a hook and loop attachment system. The handle 24 includes a hand grip 35 for grasping the handle 24 with a user's hand. One example technique for using the pad 13 is to attach the pad 13 to the handle 24, and then wet the pad 13 under running water for a few seconds. The handle 24 allows a user to position the pad 13 under the running water, and thereafter position the pad 13 on or next to a surface being treated. After contact with water, the water and the water activated material 30 of the pad 13 produce steam that flows out of the pad 13 and onto a surface being treated. If the pad 13 includes the surface treating material 31, surface treating (e.g., cleaning) solution also flows out of the pad 13 and onto a surface being treated.

While one could use the pad 13 by itself, without a handle 24, it is preferred to attach such a pad 13 to the handle 24 as shown in FIGS. 3 and 4. After use of the pad 13, the hook-and-loop type attachment system between the bottom of the mounting handle 24 and the first layer 12 of the pad 13 could be ripped apart, and the used pad 13 disposed of. A replacement pad 13 could then be abutted against the mounting handle 24 to establish another hook-and-loop connection. However, the pad 13 is not limited to a single use. For example, by controlling the amount of water added to the pad 13, unused water activated material 30 can be available in the pouch 28 for multiple uses of the pad 13. In one form, the replacement pads can be provided in perforated rolls so users can tear off a new pad in a size required for the intended use. In addition, removable attachment systems other than a hook-and-loop type attachment system can be used for attaching the pad 13 to handle 24.

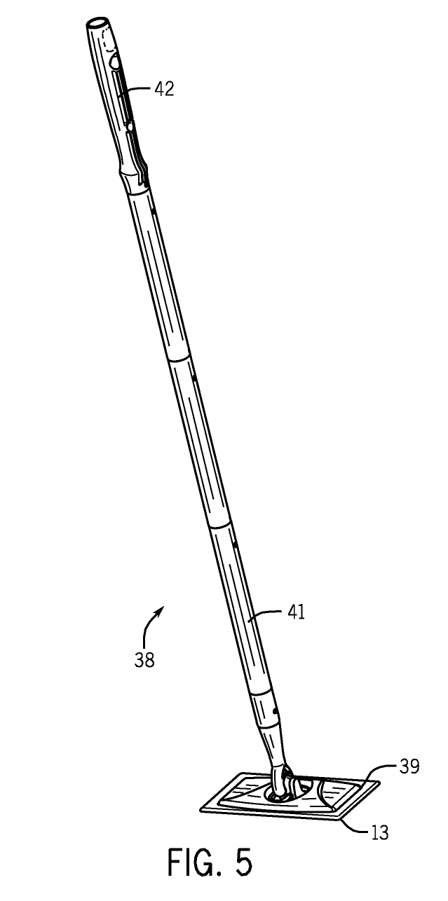

Turning to FIG. 5, there is shown a mop-type handle 38 suitable for mounting a pad 13. The mop-type handle 38 has a base 39 and an elongated shaft 41 mounted to the base 39. A grip 42 is provided at an end section of the shaft 41. The outer layer 14 of the pad 13 can be constructed to form a first part of a hook and loop attachment system and strips (not shown, but analogous to strips 32 in FIG. 3) on the base 39 can form a second part of a hook and loop attachment system for attaching the pad 13 to the mop-type handle 38. One example technique for using the pad 13 is to attach the pad 13 to the mop-type handle 38, and then wet the pad 13 with water such as in a mop bucket. The user can then use the mop-type handle 38 to direct the pad 13 against a surface (e.g., a floor or a wall) being treated. After contact with water, the water and the water activated material 30 of the pad 13 produce steam that flows out of the pad 13 and onto a surface being treated. If the pad 13 includes the surface treating material 31, surface treating (e.g., cleaning) solution also flows out of the pad 13 and onto a surface being treated.

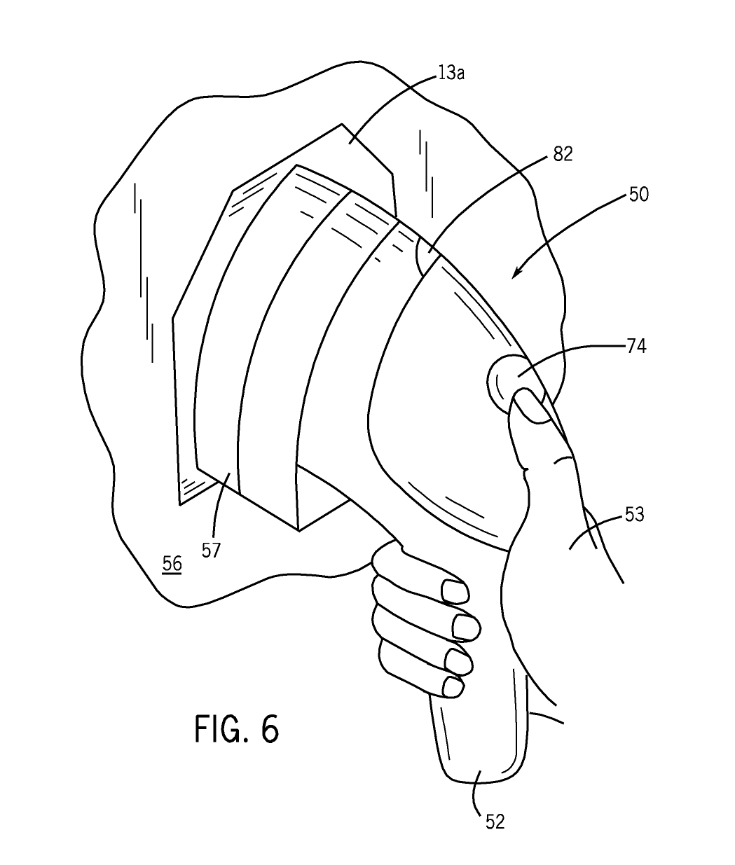

Referring now to FIGS. 6-8, an alternative pad 13a of the present invention can be used in combination with a mounting handle 50 that includes a source of water for activating water activated material in the pad 13a. The pad 13a differs from pad 13 in that pad 13a has a different shape and pad 13a does not include the surface treating material 31. The other components of pad 13a are the same as pad 13. Many different shapes for the pads 13, 13a are also possible including, without limitation, circular, elliptical. oval, polygonal, and square.

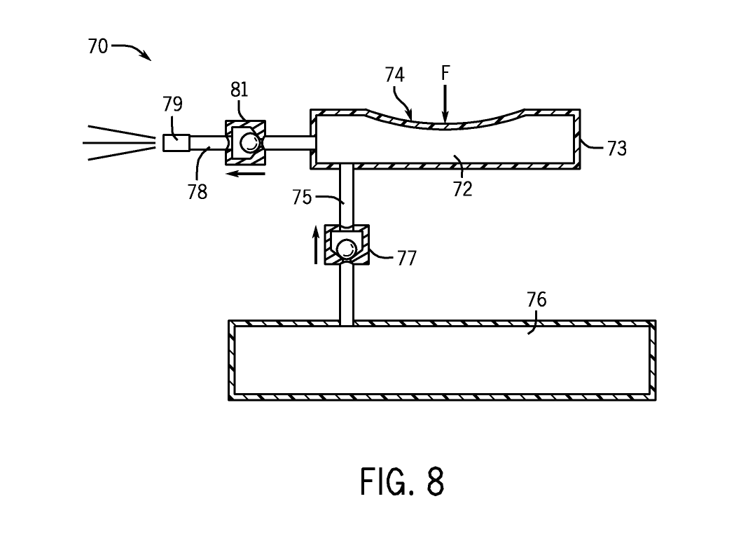

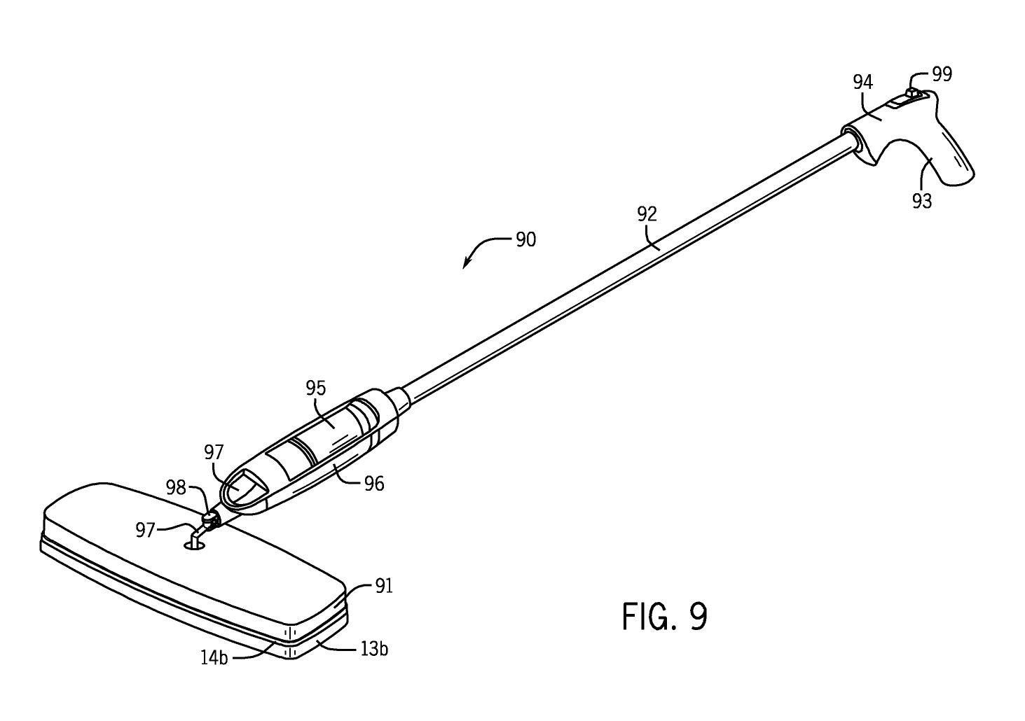

FIGS. 6 and 8 show a non-limiting example water delivery system 70 that can be incorporated into the handle 50. The water delivery system 70 includes a pump chamber 72 formed from a rigid housing 73 and a deformable elastic membrane 74 connected to the housing 73. The elastic membrane 74 (which can be mounted as a actuator button on the body of the handle 50 as shown in FIG. 6) enables the volume of a pump chamber 72 to be varied and a pumping effect is thereby accomplished. The pump chamber 72 communicates through a water supply conduit 75 with a water reservoir 76, so that water can be suctioned from the water reservoir 76 into the pump chamber 72. The water reservoir 76 can be filled with water by way of resealable opening 82 in the handle 50. Inserted in the water supply conduit 75 is a check valve 77 which permits a water flow from the water reservoir 76 to the pump chamber 72 while yet preventing a return flow of water in the opposite direction. On the downstream side the pump chamber 72, there is a water discharge conduit 78 with a water discharge nozzle 79 enabling the water to be delivered as a spray or stream. In the water discharge conduit 78, there is a check valve 81 that permits water flow from the pump chamber 72 to the nozzle 79 while preventing a return flow of water to the pump chamber 72. To deliver water, pressure exerted on the elastic membrane 74 in the direction of arrow F of FIG. 9 urges the membrane 74 into the interior of the pump chamber 72, causing the pump chamber volume to be diminished and to urge the water already contained therein through the discharge conduit 78 to the nozzle 79. Upon termination of pressure application F, the membrane 74 returns elastically to its initial position, causing the pump chamber volume to increase again. As a result, water is suctioned from the reservoir 76 through the supply conduit 75 into the pump chamber 72. The check valve 77 is in its position opening the supply conduit 75, while the check valve 81 is pulled into its position closing the discharge conduit 78. By pressing the elastic membrane 74 down again so that the volume of the pump chamber 72 diminishes again, the suctioned water in the pump chamber 72 is forced through the discharge conduit 78 and out of the nozzle 79. As a result of the increased fluid pressure in the pump chamber 72, the check valve 81 is pushed open, while the check valve 77 in the supply conduit 75 closes the conduit.

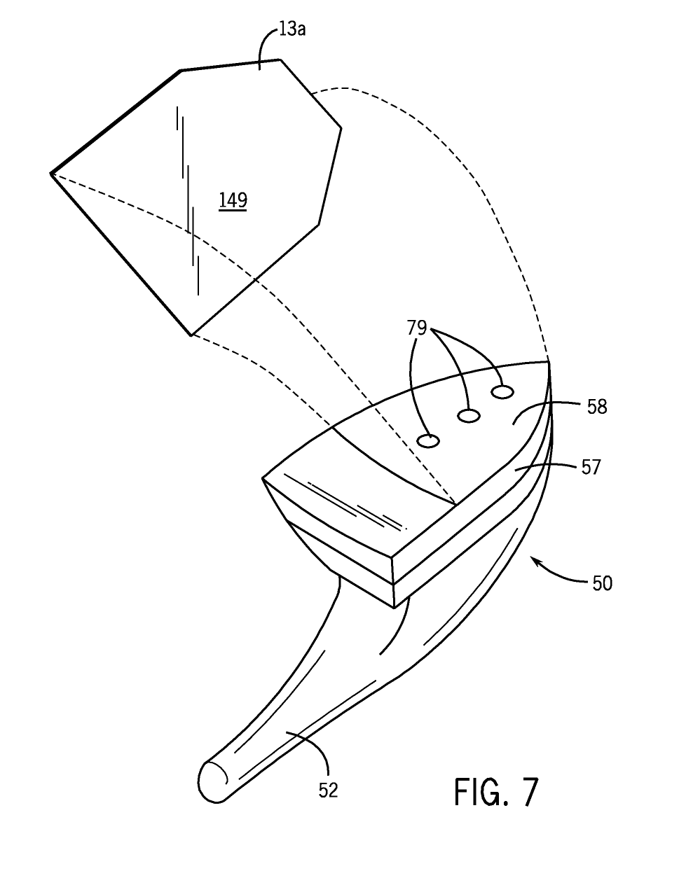

Looking at FIGS. 6 and 7, the outer layer 14a of the pad 13a can be constructed to form a first part of a hook and loop attachment system. The base 57 of the handle 50 can be constructed to form a second part of a hook and loop attachment system. The handle 50 includes a hand grip 52 for grasping the handle with a user's hand 53. One example technique for using the pad 13a is to attach the pad 13a to a base 57 of the handle 50, and then wet the pad 13a by repeatedly pressing the elastic membrane 74 of the water delivery system 70 of the handle 50. FIG. 7 shows that the outer surface 58 of the handle 50 can include multiple nozzles 79 (which can be provided at end branches of discharge conduit 78) so that the pad 13a can be wetted at multiple spaced apart locations so that steam can be generated evenly throughout the pad 13a. The water from the nozzles 79 flows through layer 14a (which is water permeable in pad 13a) and is absorbed in layer 15.

Water from layer 15 enters the pouch 28 and heat is generated by the exothermic reaction of the water and the water activated material 30. The heat is used to heat water to produce steam that flows out of the pad 13a. The water that is converted to steam can be: (i) present in the pouch 28 due to movement of water into the pouch 28; and/or (ii) absorbed in the layer 12; and/or (iii) absorbed in the layer 15; and/or (iv) absorbed in the layer 14a; and/or (v) present in the cavity 18a; and/or (vi) present in the cavity 18b. The handle 50 allows a user to position the pad 13a on or next to a surface being treated. In FIG. 6, the surface being treated is a section 56 of a garment having wrinkles that can be removed by steam generated by the pad 13a. After contact with water, the water and the water activated material 30 of the pad 13a produce steam that flows out of the pad 13a and onto the section 56 of the garment to remove the wrinkles.

Turning now to FIG. 9, there is shown a mop-type handle 90 that includes a source of water for activating water activated material in another pad 13b. The pad 13b differs from pad 13 in that pad 13b has a different shape. The other components of pad 13b are the same as pad 13. Many different shapes for the pad 13b are also possible including, without limitation, circular, elliptical. oval, polygonal, and square. The mop-type handle 90 has a base 91 connected to a hollow elongated shaft 92. A hand grip 93 is attached to an end section 94 of the shaft 92. FIG. 9 shows a non-limiting example water delivery system that can be incorporated into the handle 90. The water delivery system includes a water reservoir 95 placed in a housing 96 on the shaft 92. A water conduit 97 is in fluid communication with the reservoir 95 and an undersurface of the base 91. A valve 98 is provided in the water conduit 97 to control a flow of water from the reservoir 95 to the undersurface of the base 91. A user-operated actuating trigger 99 on the hand grip 93 is linked to the valve 98 to allow the user to open and close the valve 98 to allow water to selectively flow to the pad 13b.

The outer layer 14b of the pad 13b can be constructed to form a first part of a hook and loop attachment system. The base 91 of the handle 90 can be constructed to form a second part of a hook and loop attachment system (not shown, but analogous to strips 32 in FIG. 3). One example technique for using the pad 13b is to attach the pad 13b to the base 91 of the handle 90, and then wet the pad 13b by pressing the trigger 99 of the handle 90. The undersurface of the handle 90 can include multiple nozzles (which can be provided at end branches of water conduit 97) so that the pad 13b can be wetted at multiple spaced apart locations so that steam can be generated evenly throughout the pad 13b. The water from the nozzles flows through layer 14b (which is water permeable in pad 13b) and is absorbed in layer 15.

Water from layer 15 enters the pouch 28 and heat is generated by the exothermic reaction of the water and the water activated material 30. The heat is used to heat water to produce steam that flows out of the pad 13b. The water that is converted to steam can be: (i) present in the pouch 28 due to movement of water into the pouch 28; and/or (ii) absorbed in the layer 12; and/or (iii) absorbed in the layer 15; and/or (iv) absorbed in the layer 14b; and/or (v) present in the cavity 18a; and/or (vi) present in the cavity 18b. The handle 90 allows a user to position the pad 13b on a surface being treated (e.g., a floor). The surface being treated is cleaned and/or sanitized and/or disinfected by steam that flows out of the pad 13b and onto the surface. When the pad 13b includes a surface treating material 31 in the cavity 18b of the pad 13, the surface treating material 31 can dissolve when contacted with water to produce a surface treating (e.g., cleaning) solution that flows out of the pad 13b and onto the surface being treated. The layer 12 of the pad 13b can be used to scrub the surface that receives the cleaning solution from the pad 13b.

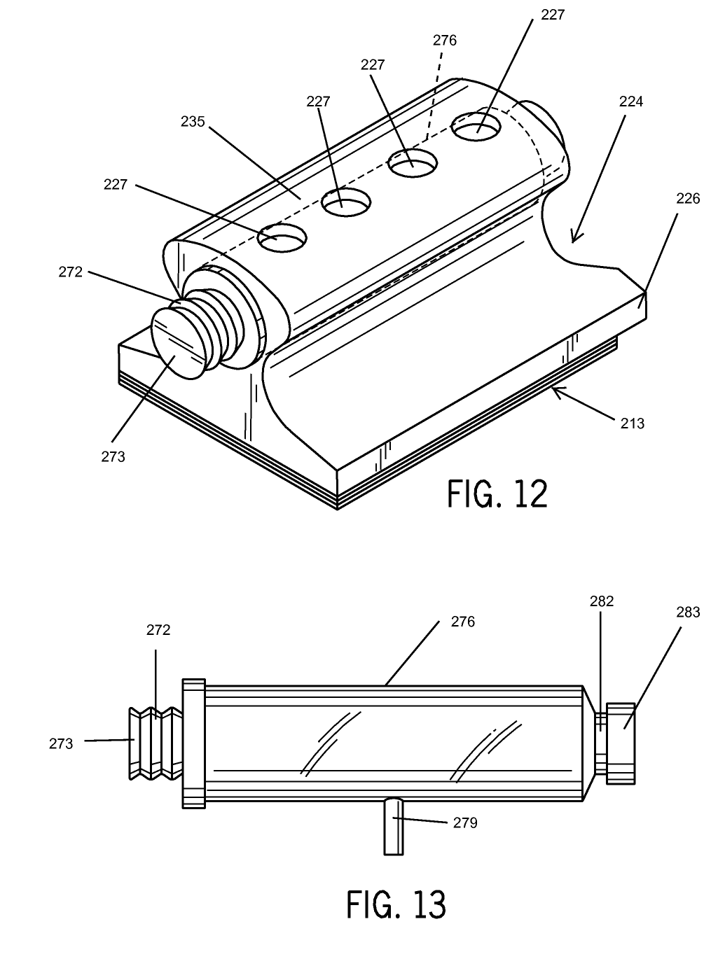

Referring next to FIGS. 12, 13 and 14, the four heating cell version of the pad 213 (or pads 13, 113) can be used in combination with a mounting handle 224. The outer layer 214 of the pad 213 can be constructed to form a first part of a hook and loop attachment system, and strips 232 on the base 226 of the handle 224 can form a second part of a hook and loop attachment system. The handle 224 includes a hand grip 235 for grasping the handle 224 with a user's hand. One example technique for using the pad 213 is to attach the pad 213 to the handle 224.

A fluid (e.g., water) delivery system is incorporated into the grip 235 of the handle 224. The fluid delivery system includes a bellows pump chamber 272 having an actuator 273 that moves the deformable elastic side walls of the bellows pump chamber 272. The actuator 273 enables the volume of a pump chamber 272 to be varied and a pumping effect is thereby accomplished. The pump chamber 272 communicates through a supply conduit with a fluid reservoir 276, so that fluid can be suctioned from the fluid reservoir 276 into the pump chamber 272. The fluid reservoir 276 can be filled with fluid by way of an externally threaded opening 282 that has a resealable internally threaded closure 283. Inserted in the supply conduit is a check valve which permits a fluid flow from the fluid reservoir 276 to the pump chamber 272 while yet preventing a return flow of fluid in the opposite direction. On the downstream side the pump chamber 272, there is a fluid discharge conduit that terminates with a fluid discharge nozzle 279 enabling the fluid to be delivered. In the fluid discharge conduit, there is a check valve that permits fluid flow from the pump chamber 272 to the nozzle 279 while preventing a return flow of fluid to the pump chamber 272. To deliver fluid, pressure exerted a few times on the actuator 273 in a direction of the nozzle 279, causing the pump chamber volume to be diminished and to urge the water already contained therein through the discharge conduit to the nozzle 279. Upon termination of pressure application, the bellows pump chamber 272 returns elastically to its initial position, causing the pump chamber volume to increase again. As a result, fluid is suctioned from the reservoir 276 through the supply conduit into the bellows pump chamber 272. The check valve is in its position opening the supply conduit, while the check valve is pulled into its position closing the discharge conduit. By pressing the actuator 273 again so that the volume of the bellows pump chamber 272 diminishes again, the suctioned fluid in the pump chamber 272 is forced through the discharge conduit and out of the nozzle 279 and out of a hollow fluid port 233 in the bottom of the handle 224. As a result of the increased fluid pressure in the bellows pump chamber 272, the check valve is pushed open, while the check valve in the supply conduit closes the conduit.

A pad 213 is attached to the handle 224 by contacting the outer surface of the layer 214 of the pad 213 to the strips 232 on the base 226 of the handle 224. The aperture 221 of the layer 214 is positioned to align with the hollow fluid port 233 in the bottom of the handle 224. After fluid in the pump chamber 272 is forced through the discharge conduit and out of the nozzle 279 and out of a central hollow fluid port 233 in the bottom of the handle 224, the fluid is delivered through the aperture 221 to the central fluid distribution site 225 (see FIG. 11). The fluid (e.g., water) runs from the fluid distribution site 225 along the heat sealed areas 217 via capillary action to the water activated material 230 speeding up the reaction time.

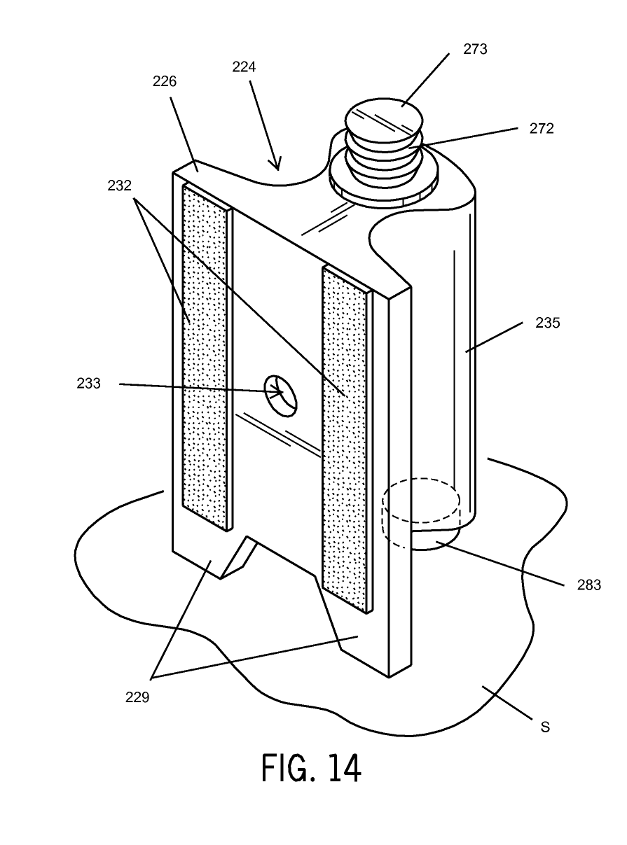

After contact with fluid, the fluid and the fluid activated material 230 of the pad 213 produce steam (or other gaseous phase) that flows out of the pad 213 and onto a surface S being treated. If the pad 213 includes the surface treating material 231, a surface treating (e.g., cleaning) solution also flows out of the pad 213 and onto a surface S being treated. FIG. 14 shows how the handle 224 can be propped up on feet 229 and closure 283 for cooling of the pad 213 (which is not shown in FIG. 14 for ease of view of the handle 224).

While one could use the pad 213 by itself, without a handle 224, it is preferred to attach such a pad 213 to the handle 224 as shown in FIG. 12. After use of the pad 213, the hook-and-loop type attachment system between the bottom of the mounting handle 224 and the layer 212 of the pad 213 could be ripped apart, and the used pad 213 disposed of. A replacement pad 213 could then be abutted against the mounting handle 224 to establish another hook-and-loop connection. However, the pad 213 is not limited to a single use. For example, by controlling the amount of water added to the pad 213, unused fluid activated material 230 can be available for multiple uses of the pad 213. In one form, the replacement pads can be provided in perforated rolls so users can tear off a new pad in a size required for the intended use. In addition, removable attachment systems other than a hook-and-loop type attachment system can be used for attaching the pad 213 to handle 224. Also, the fluid delivery system may comprise the entire handle. For example, the fluid delivery system may be shaped like handle 224 and may include strips 232 that form a second part of a hook and loop attachment system.

Alternative fluid delivery systems are also possible. For example, the fluid reservoir can have a generally hollow disc shape, and the base can be rotatably mounted to the disc shaped fluid reservoir. The base can include one or more hollow fluid ports. When more than one fluid port is used, the fluid ports can be of different diameters. The fluid reservoir includes a nozzle in its lower surface that faces the base. The nozzle can be aligned with any of the one or more hollow fluid ports by rotating the fluid reservoir with respect to the base such that the fluid flows from the fluid reservoir out of the nozzle, through the fluid port and onto a pad attached to the base. Optionally, a drip chamber can be provided upstream of the nozzle such that the rate at which fluid is provided to the nozzle is controlled. For a fluid of a given viscosity, drips from a drip chamber hole of known size will be of identical volume and the number of drips in a time period (e.g., a minute) can be counted. This version of the fluid delivery system provides for passive rather than active (i.e., pump actuated) delivery of the fluid to the pad. Also, by providing more than one fluid port with different diameters, each fluid port can provide a different amount of fluid to the pad. This provides one means for controlling the amount of heat/steam being released (e.g., low, medium, high for a device with three fluid ports of different diameters). Controlling the amount of heat/steam being released can also be achieved by controlling the dosing of the fluid activated chemistry, or by controlling both the dosing of the fluid and the dosing of the fluid activated chemistry. Controlling the amount of heat/steam being released can also be achieved via a mechanism to control output such as baffles in the pad.

Thus, in the device of FIGS. 12-14, fluid (e.g., water) is contained inside the reservoir 276 of the handle 224 of the device. The delivery of the fluid to the heating cells 219 is controlled by the end user via a bellows actuator 273 dosing a specific amount of fluid per pump. This gives users the ability to control the amount of heat produced and the longevity of the heating cells 219. Having the fluid reservoir 276 on the handle 224 also lends itself to portability. In one non-limiting example form, the fluid 276 reservoir holds 3 oz. (89 ml.), enough water to activate two pads 213.

The bellows actuator 273 dispenses on average 1.2-1.5 grams of water per pump via an internal flexible conduit. Users can be instructed to press the actuator 273 three times to activate the pad 213. Then to maintain moisture on the pad 213 and keep steam activated, users can press the button every two to three minutes. The amount of water delivered to the pad 213 is important to its functionality. Too much water can flood the reaction and stop heat from being produced.

The example pad 213 produces steam/heat for approximately ten minutes. If the pad 213 is used for less than ten minutes it may be reactivated. Users have found ten minutes to be acceptable; however, the length of time can be increased or decreased if needed based on the amount of chemistry in the pad 213. The feet 229 act as a kickstand to prop up device when hot or cooling. Holes 227 (see FIG. 12) in top of handle 224 act as a viewing window to reveal fluid level in the reservoir 276. The example pad 213 attaches via a hook and loop system with the loops on the pad 213 and the hooks on handle 224. These can be reversed with the hooks on the pad 213 and the loops on handle 224. Benefits to the users can include: “chemical-free cleaning/germ kill” and “makes cleaning quicker and easier” on hard surfaces.

One example method for using the pad 213 and the handle 224 includes: (1) fill the reservoir 276 with water; (2) attach the pad 213 to the handle 224; (3) push the actuator 273 three times to steam; (4) treat surface S by contacting the pad 213 with the surface S and moving the pad 213 over the surface S; (5) push the actuator 273 again when you need more steam; (6) repeat the surface treating of step (4); (7) prop up handle 224 on the feet 229 and the closure 283 to cool the pad 213; and (8) remove the pad 213 from the handle 224 and dispose of the pad 213.

Separate pads were evaluated in a series of experiments. One test pad had two heating cells on each side of a horizontal heat seal area, and another test pad had four heating cells arranged as in FIGS. 2B, 10 and 11. The test results showed the dosing/activation of the four heating cell pad 213 with three pumps of actuator 273 equaling 4.5 grams of water dosed to the central fluid distribution site 225 (see FIG. 11) of the pad 213 every 1.5 minutes activated the steam for ten minutes. The test results showed the dosing/activation of the two heating cell pad 213 with three pumps of actuator 273 equaling 4.5 grams of water dosed to the central fluid distribution site of the pad every 1.5 minutes activated the steam for 12.5 minutes. Also, for the two heating cell pad at 12.5 minutes, there were still small portions of the absorbent layer that remained dry. Thus, the number and location of the heating cells can be varied to vary the time period of steam generation.

Turning now to FIGS. 15-16, there is shown another mop-type handle 290 that includes a source of fluid for activating the water activated material 230 in the eight heating cell version of the pad 213. The mop-type handle 290 has a base 291 connected to a hollow elongated shaft 292. A hand grip 293 is attached to an end section of the shaft 292. A fluid delivery system can be incorporated into the handle 290. The fluid delivery system includes a fluid reservoir 295 placed in a housing 296 on the shaft 292. A fluid conduit 297 is in fluid communication with the reservoir 295 and an undersurface of the base 291. A valve assembly 298 is provided between the fluid conduit 297 and the fluid reservoir 295 to control a flow of fluid from the reservoir 295 to the undersurface of the base 291. A user-operated actuating trigger 299 on the hand grip 293 is linked to the valve assembly 298 to allow the user to open and close the valve assembly 298 to allow fluid (e.g., water) to selectively flow to the pad 213. The fluid may be gravity fed to the fluid conduit 297, or the fluid reservoir 295 may be an aerosol can with a propellant for moving the fluid to the fluid conduit 297.

The pad 213 in FIGS. 15-16 includes eight heating cells and when viewed from the top or bottom, this pad appears as two sections 213a and 213b of the pad 213 of FIG. 11 arranged in abutting side by side relationship. In this configuration, the outer layer 214 includes two apertures 221. The outer layer 214 of the pad is constructed to form a first part of a hook and loop attachment system. The base 291 of the handle 290 can be constructed to form a second part of a hook and loop attachment system (not shown, but analogous to strips 32 in FIG. 3). One example technique for using the pad is to attach the pad 213 to the base 291 of the handle 290, and then wet the pad 213 by pressing the trigger 299 of the handle 290.

The undersurface of the base 291 of the handle 290 include multiple nozzles 289 which are provided at end branches 297a and 297b of the water conduit 297 so that the pad 213 can be wetted at multiple spaced apart locations so that steam can be generated evenly throughout the pad 213. A pad 213 is attached to the base 291 of the handle 290 by contacting the outer surface of the layer 214 of the pad 213 to the base 291 of the handle 290. One aperture 221 of the layer 214 of the pad 213 is positioned to align with a nozzle at the end of the branch 297a of the fluid conduit 297. Another aperture 221 of the layer 214 of the pad 213 is positioned to align with a nozzle at the end of the branch 297b of the fluid conduit 297.

After fluid exits nozzles 289 which are provided at end branches 297a and 297b of the water conduit 297, the fluid is delivered through the apertures 221 to the central fluid distribution sites 225 of the eight cell pad. The fluid (e.g., water) runs from the fluid distribution site 225 along the heat sealed areas 217 via capillary action to the water activated material 230 speeding up the reaction time. Heat is generated by the exothermic reaction of the water and the water activated material 230. The heat is used to heat water to produce steam that flows out of the pad 213. The base 291 of the handle 290 includes four throughholes 294 that allow steam to escape through the base 291 indicating to users that the pad 213 is activated. The steam cue should be as visible as possible because of the pad's distance from the user. Therefore, the number and diameter of the throughholes 294 can be varied.

The handle 290 allows a user to position the pad 213 on a surface S being treated (e.g., a floor). The surface S being treated is cleaned and/or sanitized and/or disinfected by steam that flows out of the pad 213 and onto the surface. When the pad 213 includes a surface treating material 231 in the pad 213, the surface treating material 231 can dissolve when contacted with water to produce a surface treating (e.g., cleaning) solution that flows out of the pad 213 and onto the surface being treated. The layer 212 of the pad 213 can be used to scrub the surface S that receives the cleaning solution from the pad 213. A kickstand 288 provides a means to tilt the base 291 of the handle 290 and then prop the base 291 of the handle 290 up off the surface S while the pad 213 is hot or cooling (see FIG. 16).

Thus, in the mop-type handle 290, a fluid (e.g., water) is contained inside a fluid reservoir 295 that attaches to a shaft 292. The delivery of the water to the heating cells in the pad is controlled by the end user via trigger 299 that doses a specific amount of water per squeeze. This gives users the ability to control the amount of heat produced and the longevity of the heating cells. Having the fluid reservoir 295 on board also lends itself to portability.

The trigger 299 dispenses on average 1.2-1.5 grams of water per squeeze of the trigger 295 out of each of the two nozzles 289 in the base 291 of the handle 290. Users can be instructed to press the trigger 299 five times to activate the pad 213. Then to maintain moisture on the pad 213 and keep steam activated, users can squeeze the trigger 299 every two to three minutes. The amount of water delivered to the pad 213 can be important to its functionality. Too much water can flood the reaction and stop heat from being produced.

The pad 213 produces steam/heat for approximately ten minutes. If the pad is used for less than ten minutes, it may be reactivated. Users have found ten minutes to be acceptable however the length of time can be increased or decreased if needed based on the amount of chemistry in the pad. The four throughholes 294 in the base 291 of the handle 290 and apertures in the pad top layer 214 allow steam to escape through the base 291 of the handle 290 indicating to users the pad 213 is activated. The steam cue needs to be as visible as possible because of the pad's distance from the user.

One example method for using the pad 213 and the mop-type handle 290 includes: (1) fill the reservoir 295 with water; (2) attach the reservoir 295 to the housing 296; (3) attach the pad 213 to the base 291 of the handle 290; (4) squeeze the trigger 299 to steam; (5) treat surface S (e.g., floor) by contacting the pad 213 with the surface S and moving the pad 213 over the surface S; (6) rotate the base 291 of the handle 290 from the position in FIG. 15 to the position in FIG. 16 by pressing on the kickstand 288 with a foot; (7) prop up the base 291 of the handle 290 on the kickstand 288 to cool the pad 213 as shown in FIGS. 16; and (8) remove the cooled pad 213 from the base 291 of the handle 290 and dispose of the pad 213. Alternatively, if all of the water activated material 230 is not used up in the cleaning process, the pad 213 can be stored in a fluid tight container for later reuse starting at step (1).

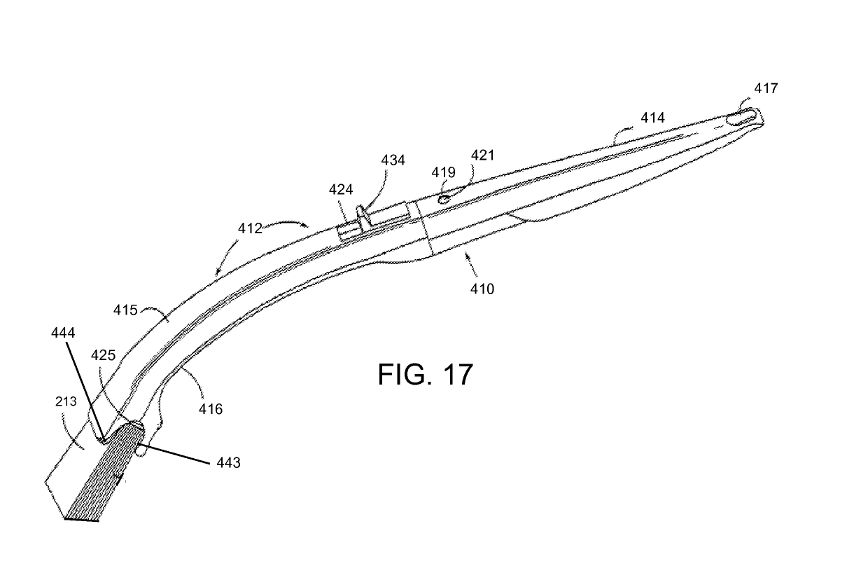

Turning now to FIG. 17, a toilet brush 410 that has the pad 213 and a multi-part wand/handle (generally 412) is shown. The wand 412 is described in more detail in U.S. Pat. No. 7,827,648 which is incorporated herein by reference. The wand 412 can be assembled from an extension 414, and upper and lower clam shell housing parts 415 and 416. The extension 414 is preferably largely hollow to reduce weight, and is formed with a hole 417 for assisting in hanging up the wand 412 (or the wand 412 with an unused pad 213 connected thereto) between uses (for example on a nail or a hook). Near the opposite end of the extension 414 is a radially extending hole 419 that is suitable to receive a corresponding snap part 421 of the housing parts 415 and 416. The housing part 415 has a radial slot 424 on one surface and an arcuate inner channel along its opposite surface. When the housing parts 415 and 416 are assembled together they form a somewhat clam shell-like housing with a hollow internal cavity communicating with a mouth outlet 425 at a lower end.

An actuator has a radially outward projecting section 434 connected by way of an elongated member to lower and upper jaws 443, 444. When the projection 434 is in one position, the lower and upper jaws 443, 444 are driven by the mouth 425 firmly against the pad 213 of the present invention. In this configuration, the lower and upper jaws 443, 444 firmly hold the pad 213. However, when a consumer pushes the projection 434 axially towards the handle mouth 425, the pad 213 will then be able to easily fall out of the jaws 443, 444 into the toilet bowl or trash can for disposal. When it is desired to reclose the jaws 443, 444 to clamp a replacement pad 213, simple axial rearward movement of the projection 434 will achieve this.

The toilet brush 410 can be used to grasp a pad 213. The pad 213 can be wetted with water by dipping the pad 213 in water in the toilet bowl. The user can then use the wand 412 to contact the pad 213 against surfaces of the toilet bowl being treated. After contact with water, the water and the water activated material 230 of the pad 213 produce steam that flows out of the pad 213 and onto the toilet bowl surface being treated. If the pad 213 includes the surface treating material 231, surface treating (e.g., cleaning) solution also flows out of the pad 213 and onto the toilet bowl surface being treated.

In an alternative method for using the toilet brush 410, the pad 213 is sealed within a gas impermeable film. A user can activate the pad 213 by opening the gas impermeable film such that air can pass through the layer 212 and through the layer 215 thereby contacting the fluid activated material 230 located in the interior space of the heating cells 219. The fluid activated material reacts exothermically when contacted with the air thereby raising the temperature of the components of the pad 213. The toilet brush 410 can be used to grasp the pad 213. The pad 213 can then be wetted with water by dipping the pad 213 in water in the toilet bowl. The user can then use the wand 412 to contact the pad 213 against a surface of the toilet bowl being treated. After contact with water, the water in the absorbent layers of the pad and the heat from the reaction produce steam that flows out of the pad 213 and onto the toilet bowl surface being treated. The increase in temperature of the components of the pad is sufficient to heat water adjacent to or in contact with one or more of the components of the pad such that the water is converted to steam.

In yet another toilet cleaning method, a pad 213 is removed from packaging and tossed into a toilet bowl. The toilet lid is closed over the toilet bowl. After contact with water, the water and the water activated material 230 of the pad 213 produce steam that flows out of the pad 213 and into the toilet bowl enclosure being treated. The toilet bowl lid keeps the cleaning and/or sanitizing and/or disinfecting steam within the toilet bowl enclosure. After a certain time period, such as ten minutes, the toilet is flushed to dispose of the pad 213. The toilet bowl is left cleaned and/or sanitized and/or disinfected.

Variations on these toilet bowl cleaning methods can be used for other enclosures. For example, a user can choose a pad 213 packaged in a gas impermeable film. The user can activate the pad 213 by opening the gas impermeable film such that air can pass through the layer 212 and through the layer 215 thereby contacting the fluid activated material 230 located in the interior space of the heating cells 219. The fluid activated material reacts exothermically when contacted with the air thereby raising the temperature of the components of the pad 213. The pad 213 can then be wetted with water. The wetted pad 213 can be placed in an enclosure such as a shower or microwave oven. After contact with water, the water in the absorbent layers of the pad and the heat from the reaction produce steam that flows out of the pad 213 into the enclosure. After a certain time period, such as ten minutes, the pad 213 is removed from the enclosure. The enclosure is left cleaned and/or sanitized and/or disinfected. Optionally, the user may wish to wipe the inside walls of the enclosure to complete the cleaning and/or sanitizing and/or disinfecting method. Alternatively, the pad 213 can be a water activated pad that is wetted with water. The wetted pad 213 can be placed in the enclosure. After contact with water, the water in the absorbent layers of the pad and the heat from the reaction produce steam that flows out of the pad 213 into the enclosure.

In another non-limiting example method, the enclosure is a storage bag containing items such as clothing. A user can choose a pad 213 packaged in a gas impermeable film. The user can activate the pad 213 by opening the gas impermeable film such that air can pass through the layer 212 and through the layer 215 thereby contacting the fluid activated material 230 located in the interior space of the heating cells 219. The fluid activated material reacts exothermically when contacted with the air thereby raising the temperature of the components of the pad 213. The pad 213 can then be wetted with water. The wetted pad 213 can be placed in the storage bag, and then the storage bag may be sealed. After contact with water, the water in the absorbent layers of the pad and the heat from the reaction produce steam that flows out of the pad 213 into the storage bag. After a certain time period, such as ten minutes, the pad 213 is removed from the storage bag. The clothing in the storage bag is left cleaned and/or sanitized and/or disinfected. Alternatively, the pad 213 can be a water activated pad that is wetted with water. The wetted pad 213 can be placed in the storage bag. After contact with water, the water in the absorbent layers of the pad and the heat from the reaction produce steam that flows out of the pad 213 into the storage bag.

Although the present invention has been described in detail with reference to certain embodiments, one skilled in the art will appreciate that the present invention can be practiced by other than the described embodiments, which have been presented for purposes of illustration and not of limitation. Therefore, the scope of the invention should not be limited to the description of the embodiments contained herein.

### INDUSTRIAL APPLICABILITY

The present invention provides a portable self-heating steam generating pad for treating hard surfaces, such as floors, walls, countertops, sinks, bathtubs, toilets and other bathroom fixtures, and/or soft surfaces, such as fabric or carpet.

All documents cited in the Detailed Description of the Invention are, in relevant part, incorporated herein by reference; the citation of any document is not to be construed as an admission that it is prior art with respect to the present invention.

What is claimed is:

1. A portable self-heating device comprising:
a heating cell having a fluid permeable wall defining an interior space of the cell and having fluid activated material located in the interior space of the cell, the material reacting exothermically when contacted with a fluid that moves through the wall into the interior space of the cell;
an outer cover having a fluid permeable section; and
an absorbent layer positioned external to the interior space of the cell,
wherein the heating cell and the absorbent layer are positioned within a cavity formed by the outer cover such that the absorbent layer contacts the heating cell.

2. The device of claim 1 wherein:
the fluid comprises liquid water, and
the material reacts exothermically when contacted with the fluid such that at least a portion of the liquid water forms steam that passes through the fluid permeable wall of the heating cell and through the fluid permeable section of the outer cover.

3. The device of claim 1 wherein:
the outer cover comprises a first layer bound to a second layer around a periphery of the first layer and a periphery of the second layer, and
the second layer is constructed to form a first part of an attachment system.

4. The device of claim 3 further comprising:
a handle constructed to form a second part of the attachment system, the handle being attached to the second layer.

5. The device of claim 4 wherein:
the handle comprises a base forming the second part of the attachment system, and a grip connected to the base.

6. The device of claim 5 wherein:
the grip is an end section of an elongated shaft.

7. The device of claim 1 further comprising:
a surface treating material incorporated into the device.

8. The device of claim 1 further comprising:
at least one additional heating cell, each additional heating cell having a fluid permeable wall defining an interior space of each additional heating cell and each additional heating cell having fluid activated material located in the interior space of each additional heating cell, the material reacting exothermically when contacted with a fluid that moves through the wall of each additional heating cell into the interior space of each additional heating cell,
wherein the absorbent layer attaches the heating cell and each additional heating cell together in spaced apart relationship.

9. A portable self-heating device comprising:
a heating cell having a fluid permeable wall defining an interior space of the cell and having fluid activated material located in the interior space of the cell, the material reacting exothermically when contacted with fluid that moves through the wall into the interior space of the cell;
an outer cover having a fluid permeable section;
an absorbent layer, the absorbent layer and the heating cell being positioned within a cavity formed by the outer cover such that the absorbent layer contacts the heating cell; and
a handle attached to the outer cover, the handle including a source of fluid and a fluid conduit in fluid communication with the source of fluid and a nozzle on a surface of the handle adjacent the outer cover, and the handle including a fluid delivery system for moving fluid from the source of fluid, through the fluid conduit, through the nozzle, through the cover, and into the absorbent layer.

10. The device of claim 9 wherein:
the fluid comprises liquid water, and
the material reacts exothermically when contacted with the fluid such that at least a portion of the liquid water forms steam that passes through the fluid permeable wall of the heating cell and through the fluid permeable section of the outer cover.

11. The device of claim 9 wherein:
the fluid delivery system includes a variable volume pump chamber in fluid communication with the source of fluid and the fluid conduit, the variable volume pump chamber moving fluid from the source of fluid, through the fluid conduit, and through the nozzle.

12. The device of claim 11 wherein:
the volume of the pump chamber is varied by an actuator on the handle.

13. The device of claim 9 wherein:
the fluid delivery system includes a valve having a closed position in which fluid cannot move from the source of fluid, through the fluid conduit, and through the nozzle, and the valve having an open position in which fluid can move from the source of fluid, through the fluid conduit, and through the nozzle.

14. The device of claim 9 wherein:
the outer cover comprises a first layer bound to a second layer around a periphery of the first layer and a periphery of the second layer, and
the second layer is constructed to form a first part of an attachment system, and
the handle is constructed to form a second part of the attachment system, the handle being attached to the second layer.

15. The device of claim 9 further comprising:
at least one additional heating cell, each additional heating cell having a fluid permeable wall defining an interior space of each additional heating cell and each additional heating cell having fluid activated material located in the interior space of each additional heating cell, the material reacting exothermically when contacted with a fluid that moves through the wall of each additional heating cell into the interior space of each additional heating cell,
wherein the absorbent layer attaches the heating cell and each additional heating cell together in spaced apart relationship.

16. The device of claim 15 wherein:
the outer cover comprises a first layer bound to a second layer around a periphery of the first layer and a periphery of the second layer,
the second layer comprises a fluid impermeable material,
the second layer includes an aperture, and
the nozzle directs fluid through the aperture and into the absorbent layer.

17. The device of claim 11 wherein:
the fluid comprises liquid water, and
the material reacts exothermically when contacted with the fluid such that at least a portion of the liquid water forms steam that passes through the fluid permeable wall of the heating cell and through the fluid permeable section of the outer cover, and
the handle comprises a base attached to the outer cover, and
the base includes at least one throughhole for passage of steam through the base.

18. A portable self-heating device comprising:
a heating cell having a fluid permeable wall defining an interior space of the cell and having fluid activated material located in the interior space of the cell, the material reacting exothermically when contacted with a fluid that moves through the fluid permeable wall into the interior space of the cell; and
an outer layer in contact with and attached to the fluid permeable wall of the heating cell, the outer layer having a fluid permeable section.

19. The device of claim 18 wherein:
the outer layer comprises an abrasive material different than a material comprising the fluid permeable wall of the heating cell.

20. The device of claim 18 wherein:
the outer layer is a fluid impermeable material different than a material comprising the fluid permeable wall of the heating cell.

21. The device of claim 18 wherein:
the fluid comprises liquid water, and
the material reacts exothermically when contacted with the fluid such that at least a portion of the liquid water forms steam that passes through the fluid permeable wall of the heating cell and through the fluid permeable section of the outer layer.

22. The device of claim 18 wherein:
the outer layer is a first part of a hook and loop attachment system.

23. The device of claim 22 further comprising:
a handle constructed to form a second part of the attachment system, the handle being attached to the outer layer.

24. The device of claim 18 further comprising:
a handle attached to the outer layer, the handle including a source of fluid and a fluid conduit in fluid communication with the source of fluid and a nozzle on a surface of the handle adjacent the outer cover, and the handle including a fluid delivery system for moving fluid from the source of fluid, through the fluid conduit, through the nozzle, through the outer layer, and into the interior space of the heating cell.

25. A method for cleaning and/or sanitizing and/or disinfecting a surface, the method comprising:
contacting the device of claim 18 with a fluid to form a wetted device; and
placing the wetted device on or adjacent the surface to contact the surface with a gas produced by the wetted device.

26. The method of claim 25 wherein:
the fluid comprises liquid water, and
the material reacts exothermically when contacted with the fluid such that at least a portion of the liquid water forms steam that passes through the fluid permeable wall of the heating cell and through the fluid permeable section of the outer layer.

27. The method of claim 26 wherein:
the surface is selected from floors, walls, countertops, sinks, bathtubs, toilets, bathroom fixtures, and inner surfaces of an enclosure.
